(19) United States
(12) Patent Application Publication
(10) Pub. No.: US 2004/0102691 A1
(43) Pub. Date: May 27, 2004

(54) Method and system for correcting images and measurements acquired by MRI

(75) Inventors: Richard Philip Mallozzi, Ballston Lake, NY (US); Ricardo Scott Avila, Clifton Park, NY (US); Paritosh Jayant Dhawale, Brookfield, WI (US); Daniel James Blezek, Niskayuna, NY (US)

Correspondence Address: GENERAL ELECTRIC COMPANY, GLOBAL RESEARCH, PATENT DOCKET RM. BLDG. K1-4A59, SCHENECTADY, NY, 12301-0008, US

(73) Assignee: General Electric Company, Niskayuna, NY (US)

(21) Appl. No.: 10/065,846

(22) Filed: Nov. 25, 2002

(51) Int. Cl. A61B 5/055

(52) U.S. Cl. 600/410; 600/416; 128/920

(57) ABSTRACT

A method and system for correcting examination images acquired by a magnetic resonance imaging (MRI) device are provided. The method comprises the steps of imaging a phantom of known structure at selected intervals to generate phantom images, analyzing the phantom images relative to images of the phantom acquired at a previous time, calculating variations between respective phantom images, and correcting the examination images and/or measurements based off the examination images using the calculated variations between phantom images. The system comprises an imaging device for acquiring images of a subject and the phantom and an image processor adapted to correct the examination images based on the calculated variations between respective phantom images.

### BACKGROUND OF INVENTION

The present invention relates to correction and calibration methods for use in Magnetic Resonance Imaging (MRI). More particularly, the present invention relates to methods of calibrating and correcting for errors in volumetric measurements acquired for longitudinal examinations where the errors are attributable to variations due to MRI scanner hardware.

It is well known to employ MRI for visualization of internal anatomical structures, such as brain imaging. Brain imaging, particularly for use in brain volume measurements, is of particular interest for studying neurodegenerative diseases (NDD), such as Alzheimer's disease and Parkinson's Diseases. Brain volume measurements have great promise for tracking the progression of a number of NDDs. For example, normal aging brain atrophy due to natural biological variation is about a 2.5% decrease per decade (¼% per year) but the rate of atrophy increases in subjects with Alzheimer's Disease. It has been shown that the brain volume of clinically-diagnosed Alzheimer's patients decreases by about 2-5% every year. Thus, brain volume measurements provide a measurable correlation available to track Alzheimer's Disease. In addition, volumetric measurements on regions within the brain, such as the hippocampus and entorhinal cortex, have shown these structures exhibit measurable atrophy well before observable cognitive symptoms appear. Hence volumetric measurements on the brain could be an important method for the early diagnosis and prediction of the disease. The diagnosis of other brain diseases such as tumors and edema also rely on brain volume measurement.

However, MRI volumetric measurements are susceptible to errors attributed to the MRI scanner hardware and introduce a margin of error that may not be acceptable for brain volume measurements. Such factors include gradient amplifier drift, shim drift, room temperature changes and imprecise calibration. There are variations within a single scanner (i.e. intra-scanner variability) making volumetric measurements and there may be considerable variation in measurements made between different scanners (i.e. inter-scanner variability). It is desirable to reduce such errors as much as possible, particularly when imaging is performed for the purpose of measuring brain volumes for tracking certain neurodegenerative diseases, such as Alzheimer's Disease and Parkinson's Disease, so that changes in brain volume measurements are isolated from variations due to the scanner.

In addition, MRI hardware is often changed or upgraded in a piece-wise fashion, such as with new rf coils, new gradient coils, or new system electronics. Such upgrades can adversely affect volumetric measurements. A technique is needed to address hardware inconsistency introduced by such hardware changes as well.

A number of NDDs, such as Alzheimer's and Parkinson's Diseases, are associated with a change in volume of various regions of the central nervous system, for example, the hippocampus amygdala region of the brain. However, measuring the volumes of sub-structures within the brain with sufficient precision is a significant challenge given the variations attributable to the scanner. Thus, such variations due to the scanner need to be minimized in order to avoid introducing additional error. As discussed above, changes in volume can be quite small, from fractions of a percent per year to a few percent per year. Further, there is a need to perform successive examinations to measure the brain volume of a given patient to track progression of the disease. Successive examinations of a given patient are often referred to as longitudinal examinations. It is desirable to correct for scanner-attributed errors in longitudinal measurements. Thus, measuring structures with high precision and maintaining the stability of measurements over periods of months and years is very challenging for MRI. Such measurements are often corrupted by drifts in the scanner hardware, or by abrupt changes such as recalibration or installation of new hardware.

Thus, what is needed is a method of correcting for scanner related errors in images and/or volumetric measurements acquired by MRI.

### SUMMARY OF INVENTION

In a first aspect, a method for correcting examination images acquired by a magnetic resonance imaging (MRI) device is provided. The method comprises the steps of imaging a phantom of known structure at selected intervals to generate phantom images, analyzing the phantom images relative to images of the phantom acquired at a previous time, calculating variations between respective phantom images, and correcting the examination images and/or measurements based off the examination images using the calculated variations between phantom images.

In a second aspect, a system for performing Magnetic Resonance Imaging (MRI) examinations is provided. The system comprises an imaging device, such as a MRI scanner, for acquiring images of regions of interest within a subject and a phantom of known structure, as described in greater detail above. Further provided is an image processor adapted to analyze images of the phantom and calculate scanner related variations and further adapted to automatically correct images of regions of interest within the subject based on the calculated variations.

### BRIEF DESCRIPTION OF DRAWINGS

The features and advantages of the present invention will become apparent from the following detailed description of the invention when read with the accompanying drawings in which:

FIG. 1 illustrates a simplified block diagram of a Magnetic Resonance Imaging system to which embodiments of the present invention are useful;

FIG. 2 is an exemplary embodiment of a phantom for use in embodiments of the present invention;

FIG. 3 is an exemplary flow diagram of a correction method for correcting volume measurements acquired by MRI;

FIG. 4 is a second exemplary flow diagram of a correction method for correcting images and volume measurements acquired by MRI; and,

FIG. 5 is a simplified illustration of images of a phantom at various times during methods of the present invention.

### DETAILED DESCRIPTION

MRI scanners, which are used in various fields such as medical diagnostics, typically use a computer to create images based on the operation of a magnet, a gradient coil assembly, and a radio frequency coil(s). The magnet creates a uniform main magnetic field that makes nuclei, such as hydrogen atomic nuclei, responsive to radio frequency excitation. The gradient coil assembly imposes a series of pulsed, spatial magnetic fields upon the main magnetic field to give each point in the imaging volume a spatial identity corresponding to its unique set of magnetic fields during the imaging pulse sequence. The radio frequency coil(s) creates an excitation frequency pulse that temporarily creates an oscillating transverse magnetization that is detected by the radio frequency coil and used by the computer to create the image.

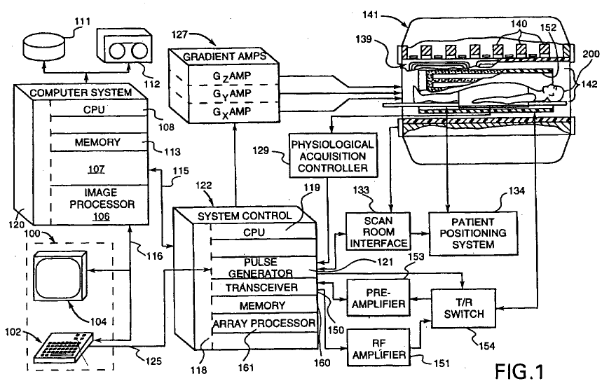

FIG. 1 illustrates a simplified block diagram of a system for producing images in accordance with embodiments of the present invention. In an embodiment, the system is a MR imaging system which incorporates the present invention. The MRI system could be, for example, a GE-Signa MR scanner available from GE Medical Systems, Inc., which is adapted to perform the method of the present invention, although other systems could be used as well.

The operation of the MR system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules that communicate with each other through a backplane. These include an image processor module 106, a CPU module 108, and a memory module 113, known in the art as a frame buffer for storing image data arrays. The computer system 107 is linked to a disk storage 111 and a tape drive 112 for storage of image data and programs, and it communicates with a separate system control 122 through a high speed serial link 115.

The system control 122 includes a set of modules connected together by a backplane. These include a CPU module 119 and a pulse generator module 121 which connects to the operator console 100 through a serial link 125. It is through this link 125 that the system control 122 receives commands from the operator which indicate the scan sequence that is to be performed. The pulse generator module 121 operates the system components to carry out the desired scan sequence. It produces data that indicate the timing, strength, and shape of the radio frequency (RF) pulses that are to be produced, and the timing of and length of the data acquisition window. The pulse generator module 121 connects to a set of gradient amplifiers 127, to indicate the timing and shape of the gradient pulses to be produced during the scan. The pulse generator module 121 also receives subject data from a physiological acquisition controller 129 that receives signals from a number of different sensors connected to the subject 200, such as ECG signals from electrodes or respiratory signals from a bellows. And finally, the pulse generator module 121 connects to a scan room interface circuit 133 (which receives signals from various sensors associated with the condition of the subject 200) and the magnet system. It is also through the scan room interface circuit 133 that a positioning device 134 receives commands to move the subject 200 to the desired position for the scan.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a whole-body RF coil 152. Volume 142 is shown as the area within magnet assembly 141 for receiving subject 200 and includes a patient bore. As used herein, the usable volume of a MRI scanner is defined generally as the volume within volume 142 that is a contiguous area inside the patient bore where homogeneity of main, gradient and RF fields are within known, acceptable ranges for imaging. A transceiver module 150 in the system control 122 produces pulses that are amplified by an RF amplifier 151 and coupled to the RF coil 152 by a transmit/receive switch 154. The resulting signals radiated by the excited nuclei in the subject 200 may be sensed by the same RF coil 152 and coupled through the transmit/receive switch 154 to a preamplifier 153. The amplified MR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate RF coil (for example, a head coil or surface coil) to be used in either transmit or receive mode. As used herein, “adapted to”, “configured” and the like refer to mechanical or structural connections between elements to allow the elements to cooperate to provide a described effect; these terms also refer to operation capabilities of electrical elements such as analog or digital computers or application specific devices (such as an application specific integrated circuit (ASIC)) that is programmed to perform a sequel to provide an output in response to given input signals.

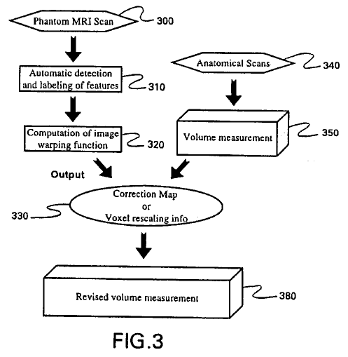

The MR signals picked up by the RF coil 152 are digitized by the transceiver module 150 and transferred to a memory module 160 in the system control 122. When the scan is completed and an entire array of data has been acquired in the memory module 160, an array processor 161 operates to Fourier transform the data into an array of image data. These image data are conveyed through the serial link 115 to the computer system 107 where they are stored in the disk memory 111. In response to commands received from the operator console 100, these image data may be archived on the tape drive 112, or they may be further processed by the image processor 106 and conveyed to the operator console 100 and presented on the display 104. Image processor 106 is further adapted to perform the image processing techniques which will be in greater detail below and with reference to FIGS. 3-5. It is to be appreciated that a MRI scanner is designed to accomplish field homogeneity with given scanner requirements of openness, speed and cost.

All data gathered from multiple scans of the patient is to be considered one data set. Each data set can be broken up into smaller units, either pixels or voxels. When the data set is two-dimensional, the image is made up of units called pixels. A pixel is a point in two-dimensional space that can be referenced using two-dimensional coordinates, usually x and y. Each pixel in an image is surrounded by eight other pixels, the nine pixels forming a three-by-three square. These eight other pixels, which surround the center pixel, are considered the eight-connected neighbors of the center pixel. When the data set is three-dimensional, the image is displayed in units called voxels. A voxel is a point in three-dimensional space that can be referenced using three-dimensional coordinates, usually x, y and z. Each voxel is surrounded by twenty-six other voxels. These twenty-six voxels can be considered the twenty-six connected neighbors of the original voxel.

For embodiments of the present invention, subject 200 is a given patient that is imaged for a first examination and then at least one successive examination, typically at a later time, in order to measure the progression of a given neurodegenerative disease (NDD) such as Alzheimer's or Parkinson's Disease. However, the embodiments would also be applicable to imaging sessions for other diseases where it would be important or desirable to correct for image disturbance due to the imaging device. Further, the embodiments would also be applicable to imaging sessions using two or more different imaging devices.

In a first embodiment, a method for correcting examination images acquired by a magnetic resonance imaging (MRI) device comprises imaging a phantom of known structure at selected intervals to generate phantom images, analyzing the phantom images relative to images of the phantom acquired at a previous time, calculating variations between respective phantom images, and correcting the examination images and/or measurements based off the examination images using the calculated variations between phantom images. The method is desirably automatic and does not require the operator of the imaging device to initiate the correction methods. In practice, prior to scanning subject 200, the given patient, the phantom is imaged and the correction process is initiated as part of imaging or scanning the patient. The interval for imaging the phantom would be determined by the stability of a particular scanner. Such stability could be measured with the phantom. Alternatively, a specification could be generated to which all scanners would minimally conform for longitudinal stability. In any case, this time period is likely to be from once per day to once every few weeks.

The correction process involves imaging a phantom of known structure. An image, or a set of images, is taken of the phantom. The phantom contains structures within it that can be precisely located, such as small spheres. The apparent location of these structures in the image will change if certain types of scanner drift or errors occur.

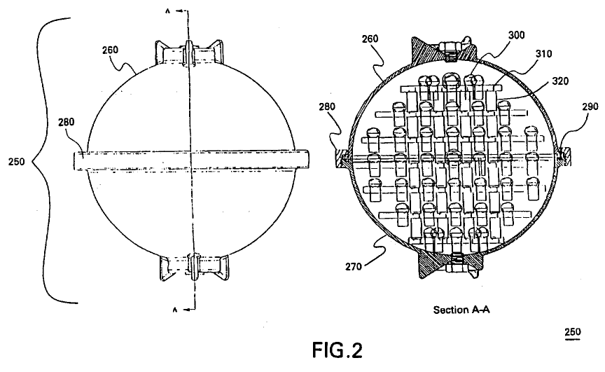

Referring to FIG. 2, there is shown an exemplary embodiment of a phantom 250 to which embodiments of the present invention are applicable (two view are shown, the cross-section view is taken at along A-A). Phantom 250 comprises an outer structure, shown in FIG. 2 as spherical in shape and comprising an upper hemisphere 260, a lower hemisphere 270, a locking ring 280 for coupling the upper and lower hemispheres and an o-ring 290 for sealing the assembly. Contained within the phantom 250 are a plurality of smaller substructures, shown as spherical objects 300. The phantom further comprises a plurality of substructures embedded in, or otherwise contained within, the outer structure of the phantom. The size of the outer structure (the assembly shown in FIG. 2 comprising the upper and lower hemisphere, 260 and 270 respectfully, and the locking ring 280) is chosen to represent the field of view of the image. The small spheres 300 and the larger sphere (260, 270, and 280, hereinafter referred to as the phantom structure) are filled with or constructed of materials that have different signal strengths in an MR image. For example, in the exemplary embodiment, there are 171 small spheres containing copper sulfate (CuSO4) and the outer structure is filled with distilled water thereby providing contrast between the small spheres and the water. The small spheres 300 are held in place by some type of support structure that appears dark in MR images (e.g. solid plastic structures 310 and 320 are constructed to hold the spheres in a given pattern) and the support structure is constructed of material that has a good magnetic susceptibility match to water to prevent distortion (e.g. plastic). The small spheres are a preferred shape because such a shape lends itself to a highly precise identification of the location of the center of the sphere. The size and number of the substructures can vary depending on different correction factors needed. The substructures are helpful for more precise and accurate identification of a given location within an image. For example, more substructures help with corrections of non-linear and higher order errors, in that the substructures, when detected by the imaging device, provide a larger body of information available for extracting higher-order error in the fields. The size of the substructures or smaller spheres is chosen to correspond to the level of voxel correction desired. Typically, a voxel measures approximately 1-2 mm³. It may be desirable to have the capability to correct at a sub-voxel level in which a larger sphere is more desirable. Typically for good sub-voxel localization, the sphere diameter would comprise several voxels. In this exemplary embodiment, the size of the substructures are preferably about 0.5 mm to about 30 mm in diameter for correcting brain volume measurements calculated from acquired MRI images. The outer structure is chosen to be spherical or ellipsoidal to reduce distortion caused by the mismatch in magnetic susceptibility between the phantom and the surrounding environment. The choice of a sphere or ellipsoid for the phantom shape renders the distortion of the static magnetic field uniform within that sphere or ellipsoid. Such a change will generally have an effect only of translating the image, rather than any more complex distortion. It is to be appreciated that one skilled in the art may choose other shapes that would similarly isolate and reduce image distortion. The spherical shape is presented as an exemplary embodiment.

In order to analyze the phantom images relative to previous images of the phantom, the plurality of substructures in each of the images are located and assigned. The preferred embodiment is an algorithm that performs this location automatically. There are various known ways to detect features in a given image, in this case the substructures or smaller spheres 300. One such embodiment would automatically determine an intensity threshold based on the collected image data. The threshold would be applied to the image resulting in a pixel labeling identifying structures of interest and background. A grouping algorithm is employed to collect connected pixels into connected components. Each component is processed in turn. For each component, the center of mass is determined using standard first moment techniques on the combination of pixel spatial position and intensity level. The center of mass calculation provides a robust estimate of the true structure center.

Once the substructures 300 are located, the correction method calculates variations between the phantom and previous images of the phantom. The location of the structures in the phantom can be compared to their location during previous scans as described above with reference to the analyzing step, or to their known position. Relative changes in position or location are calculated to yield information about the errors in the image of interest (anatomical image).

A preferred embodiment of the calculation of variation is to use the measurements of the discrete structures within phantom to determine a continuous function that describes the warping of the phantom. Such a continuous function would effectively be a means of interpolating between the measured values at the discrete locations of the measured structures within the phantom.

One such embodiment would be a fit of some set of continuous basis functions to the measured errors or changes in the phantom structures. The choice of number of such basis functions used would depend upon the number and distribution of discrete structures with the phantom.

Another embodiment would be to assume a physical model for the distortion, such as a compressible fluid. Such a model may impose certain constraints on the nature of the distortion, which could be a desired feature of the interpolation scheme.

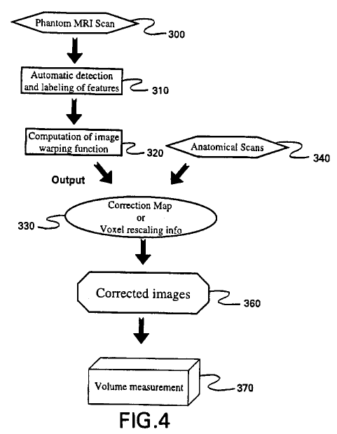

Once the variations between phantom images are analyzed and determined, the method of the present invention corrects the examination images or, alternatively, measurements (for example, volumes) based on the images using the calculated variations. Referring to FIGS. 3 and 4, there are shown two methods for correcting the examination images or measurements. In both figures, the phantom is imaged at 300 in any of the ways described above. At 310, there is an automatic detection and location of features (the smaller spheres 300 of FIG. 2) within the images as described above in analyzing the location of the substructures of the phantom. There is a computation of image warping at 320, wherein warping corresponds to the variation between phantom images. There are multiple ways to implement a correction to the desired measurements (volumes).

Referring first to FIG. 3, the images of interest (anatomical images from 340) are left in their original state, and the warping information from the phantom measurement could be used to correct volumes (acquired at 350) measured on the image of interest. In this embodiment, the volume measurements are corrected at step 380 using a scaling correction map for each voxel (330) and adjusting the reported volume of every voxel. A correction map or voxel resealing information is calculated at 330 for using in correcting the images from anatomical scans acquired at 340.

Referring to FIG. 4, the images are corrected to undo the error introduced by the imaging device and then the volume measurements are generated based on the corrected images. In this embodiment and referring further to FIG. 4, a correction map is calculated at step 330. This map gives the warping that needs to be applied to the anatomical image to undo the error introduced by the hardware. This correction map would be used, for example, to warp the anatomical image to better represent how it would appear without the introduction of hardware-induced errors. Such warping would preferably involve a re-sampling of the image to maintain continuous warping. After this correction of the image, the measurement of interest is performed, such as a volume measurement 370. The general feature of such a correction would be to change the original image, through spatial warping and possible intensity correction, to give a new image that is believed to more accurately reflect the image that would have been measured without errors or changes in the scanner hardware or processing.

Such a phantom measurement can be used for corrections other than volume. One could measure the signal-to-noise or the brightness of structures within the phantom and use them to modify brightness or signal-to-noise in the image of interest (anatomical image).

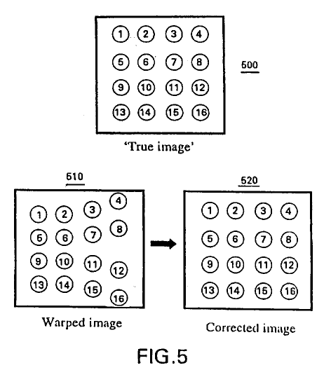

Referring to FIG. 5, there is shown a simplified illustration of a true image 500 having substructures 1-16 at a given grid location. In an exaggerated illustration, a warped or distorted image 510 shows that substructures 1-16 have strayed from their original respective locations. After applying the correction methods described above, the corrected image shows that substructures 1-16 have corrected locations corresponding to substantially their respective original locations.

The methods described are particularly useful for longitudinal examinations. Thus, a method for performing longitudinal examinations of a set of anatomical structures is provided. The method comprises the steps of imaging the anatomical structures; performing an automatic correction process (as described above using a phantom) to correct for variations due to the imaging device or process, measuring selected regions of interest within the anatomical structures, and repeating the imaging, running and measuring steps for any number of successive examinations in order to track the progression of a given disease.

There are many known methods of measuring selected regions. One could, for example, draw lines manually to delineate a region of interest and then count the number of voxels within. Another method is to use point sampling together with a skilled user to determine the location of the sampled points. Another method is to start with a “seed” point in the image and use a region-growing technique to determine which of the nearby points are also in the region of interest. Iterating this process, starting with points identified in the previous iteration, eventually yields a connected set of points in a region of interest. Still another method is to use the relaxation properties of the tissue to identify what general type of tissue it is, and hence measure the quantity of tissue of a desired type.

The correction method discussed in this patent applies to a wide variety of different techniques for measuring the selected regions of interest.

Repeating the steps above, at a later time, yields a longitudinal measurement. The correction technique will adjust the measurement for errors caused by the scanner that are different from one iteration to another. In addition, this correction technique can be used to adjust the measurement errors caused from a change in hardware configuration. The application method would be identical to that discussed above. Such measurements are of use in the diagnosis, tracking, and prediction of neurological disorders.

The methods described above may further comprise a co-registration step to register successive images. Some measurement techniques will include a step that co-registers different images to increase the accuracy or functionality of the measuring technique. This is an optional part of this technique.

In a further embodiment, the correction process corrects due to different scanners where a given patient or subject is imaged on different imaging devices or scanners. The phantom would be imaged in the two different scanners and the resulting images would be used to calculate variations for use in correcting anatomical images acquired of the subject. The input for the scanner is the data and/or correction information from a scan of the calibration phantom. This information can be used in an automated way to make the corrections of the measurements of interest.

In a further aspect, a system for performing Magnetic Resonance Imaging (MRI) examinations is provided. The system comprises an imaging device, such as a MRI scanner, for acquiring images of regions of interest within a subject and a phantom of known structure, as described in greater detail above. Further provided is an image processor adapted to analyze images of the phantom and calculate scanner related variations and further adapted to automatically correct images of regions of interest within the subject based on the calculated variations.

As described above, the images of the regions of interest are acquired at a first imaging session and at least one successive imaging session for tracking progression of a given disease, such as the neurodegenerative diseases described in detail that rely on volumetric measurements as a correlation to disease progression. Further, in image processor is the image processor is adapted to automatically correct the images or, alternatively, the measurements derived from the images of the regions of interest. The system is particularly adapted to perform longitudinal examinations of a given patient to track progression of the given disease by correcting for errors attributable to imaging device.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

1. A method for correcting at least one of examination images and measurements acquired by a magnetic resonance imaging (MRI) device comprises:
imaging a phantom of known structure at selected intervals to generate phantom images;
performing automatic analysis of the phantom images relative to images of the phantom acquired at a previous time;
calculating variations between respective phantom images; and,
correcting the at least one of examination images and measurements using the calculated variations between phantom images.

2. The method of claim 1 wherein the correcting step is automatically initiated within an image processor of the MRI device.

3. The method of claim 1 wherein the phantom comprises an outer structure and a plurality of substructures contained within the outer structure, wherein the outer structure and substructures are constructed of respective materials having different signal strengths in a MR image.

4. The method of claim 1 wherein the examination images are acquired at a first imaging session and at least one successive imaging session for tracking progression of a given disease.

5. The method of claim 4 wherein the disease is a neurodegenerative disease and the examination images are used to generate volume measurements of a brain.

6. The method of claim 3 wherein the performing step comprises determining respective locations of the substructures within the respective phantom images.

7. The method of claim 6 wherein the calculating step calculates relative changes in the locations of the substructures to provide correction information.

8. The method of claim 1 wherein the correcting step generates corrected examination images and volume measurements are generated based on the corrected examination images.

9. The method of claim 1 wherein the correcting step corrects volume measurements using a scaling correction map for adjusting the volume measurements.

10. A method for performing longitudinal examinations of a set of anatomical structures comprising:
imaging the structures with at least one imaging device;
performing an automatic correction process to correct for variations due to the imaging device or process;
measuring selected regions of interest within the anatomical structures; and
repeating the imaging, performing and measuring steps for at least one successive examination for tracking the measured regions of interest.

11. The method of claim 10 wherein the correction process comprises the steps of:
imaging a phantom of known structure at selected intervals to generate phantom images;
analyzing the phantom images relative to images of the phantom acquired at a previous time;
calculating variations between respective phantom images; and,
correcting at least one of examination images of the anatomical structures and measurements using the calculated variations between phantom images.

12. The method of claim 11 wherein the phantom comprises an outer structure and a plurality of substructures contained within the outer structure, wherein the outer structure and substructures are constructed of respective materials having different signal strengths in a MR image.

13. The method of claim 10 wherein the regions of interest are substructures within the brain.

14. The method of claim 10 further comprises a co-registration step to register successive images of the anatomical structures.

15. The method of claim 10 wherein the correction process corrects due to different imaging devices.

16. A system for performing Magnetic Resonance Imaging (MRI) examinations comprising:
an imaging device for acquiring images of regions of interest within a subject and a phantom of known structure; and,
an image processor adapted to analyze images of the phantom and calculate scanner related variations and further adapted to automatically correct at least one of images of regions of interest and measurements of the regions of interest within the subject based on the calculated variations.

17. The system of claim 16 wherein the phantom comprises an outer structure and a plurality of substructures contained within the outer structure, wherein the outer structure and substructures are constructed of respective materials having different signal strengths in a MR image.

18. The system of claim 16 wherein the images of the regions of interest are acquired at a first imaging session and at least one successive imaging session for tracking progression of a given disease.

19. The system of claim 18 wherein the disease is a neurodegenerative disease and the images of the region of interest are used to generate volume measurements of a brain.

20. The system of claim 16 wherein the image processor is further adapted to automatically correct measurements derived from the images of the regions of interest.
